SEQUENCE LISTING (19 sequences)

SEQ ID NO: 1 - protein, 28 aa, Artificial
  Synthetic kinase substrate
LRRLSLGLRRLSLGLRRLSLGLRRLSLG

SEQ ID NO: 2 - protein, 17 aa, Artificial
  Synthetic kinase substrate
LDDRHDSGLDSMKDEEY

SEQ ID NO: 3 - protein, 10 aa, Artificial
  Synthetic kinase substrate
KKLNRTLSVA

SEQ ID NO: 4 - protein, 23 aa, Artificial
  Synthetic kinase substrate
KKKVSRSGLYRSPSMPENLNRPR

SEQ ID NO: 5 - protein, 15 aa, Artificial
  Synthetic kinase substrate
FLAKSFGSPNRAYKK

SEQ ID NO: 6 - protein, 11 aa, Artificial
  Synthetic kinase substrate
RSRHSSYPAGT

SEQ ID NO: 7 - protein, 18 aa, Artificial
  Synthetic kinase substrate
ISDELMDATFADQEAKKK

SEQ ID NO: 8 - protein, 32 aa, Artificial
  Synthetic kinase substrate
KEAKEKRQEQIAKRRRLSSLRASTSKSGGSQK

SEQ ID NO: 9 - protein, 10 aa, Artificial
  Synthetic kinase substrate
KKLNRTLSVA

SEQ ID NO: 10 - protein, 15 aa, Artificial
  Synthetic kinase substrate
KVEKIGEGTYGVVYK

SEQ ID NO: 11 - protein, 15 aa, Artificial
  Synthetic kinase substrate
RSRSRSRSRSRSRSR

SEQ ID NO: 12 - protein, 11 aa, Artificial
  Synthetic kinase substrate
YLRRRLSDSNF

SEQ ID NO: 13 - protein, 15 aa, Artificial
  Synthetic kinase substrate
RKKFGESKTKTKEFL

SEQ ID NO: 14 - protein, 13 aa, Artificial
  Synthetic kinase substrate
KKRPQRATSNVFA

SEQ ID NO: 15 - protein, 10 aa, Artificial
  Synthetic kinase substrate
KKLNRTLSVA

SEQ ID NO: 16 - protein, 15 aa, Artificial
  Synthetic kinase substrate
FLAKSFGSPNRAYKK

SEQ ID NO: 17 - protein, 9 aa, Artificial
  Synthetic kinase substrate
RFRRSRRMI

SEQ ID NO: 18 - protein, 13 aa, Artificial
  Synthetic kinase substrate
GRPRTSSFAEGKK

SEQ ID NO: 19 - protein, 14 aa, Artificial
  Synthetic kinase substrate
KKISGRLSPIMTEQ

(19) United States
(12) Patent Application Publication
(10) Pub. No.: US 2010/0063085 A1
(43) Pub. Date: Mar. 11, 2010

(54) METHOD OF TREATING LEARNING IMPAIRMENT IN DOWN'S SYNDROME SUBJECTS

(75) Inventor: Philip Cohen, Dundee (GB)

Correspondence Address: MYERS BIGEL SIBLEY & SAJOVEC, PO BOX 37428, RALEIGH, NC, 27627, US

(73) Assignee: University Court of The University of Dundee

(21) Appl. No.: 12/208,677

(22) Filed: Sep. 11, 2008

(51) Int. Cl. A61K 31/437; A61P 25/00

(52) U.S. Cl. 514/292

(57) ABSTRACT

The present invention provides compounds for use in the preparation of pharmaceutical formulations and medicaments, which alleviate learning and/or mental impairment in people suffering from Down's syndrome.

### FIELD OF THE INVENTION

The present invention provides compounds for use and methods of alleviating learning and/or mental impairment in people suffering from Down's syndrome.

### BACKGROUND OF THE INVENTION

People with Down's syndrome have an extra chromosome (number 21) in some or all of their cells. This results in physical and mental characteristics which often includes learning difficulties, common facial features and heart problems. Approximately 1 in 1000 children born in the UK are affected by Down's syndrome.

Children with Down's syndrome usually learn to walk, talk, read and write, but more slowly than other children of their age and ultimate IQ is generally below average.

Screening tests are available that can assist parents to assess the risk of their unborn child having Down's syndrome and some parents may decide to terminate the pregnancy if their baby has Down's syndrome.

Babies with Down's syndrome are usually diagnosed in the first few days after birth. However, there are currently no drug therapies for alleviating symptoms, such as the mental and learning impairment associated with Down's syndrome.

An object of the present invention is to obviate and/or mitigate mental and/or learning impairment in people with Down's syndrome.

A further object of the invention is to provide compounds suitable for use in children with Down's syndrome to help treat the mental and/or learning impairment associated with Down's syndrome.

The present invention is based in part on work carried out by the present inventors in relation to the protein kinase DYRK1a (Woods Y L, Rena G, Morrice N, Barthel A, Becker W, Guo S, Unterman T G, Cohen P. Biochem. J. 2001, 355, 597-607 The kinase DYRK1A phosphorylates the transcription factor FKHR at Ser329 in vitro, a novel in vivo phosphorylation site; Woods Y L, Cohen P, Becker W, Jakes R, Goedert M, Wang X, Proud C G. Biochem. J. 2001, 355, 609-615 The kinase DYRK1A phosphorylates protein-synthesis initiation factor elf2Bε at Ser 539 and the microtubule-associated protein tau at Thr 212: potential role for DYRK as a glycogen synthase kinase 3-priming kinase). Since those with Down's tend to have a problem converting phenylalanine into tyrosine, it is possible that DYRK (dual-specificity tyrosine—regulated kinase) may have some involvement in regulating this reaction. Moreover, DYRK1a is known to map to the “Down's syndrome critical region” (Guimera J, Casas C, Pucharos C, Solans A, Domenech A, Planas A M, Ashley J, Lovett M, Estivill X, Pritchard M. Hum Mol Genet. 1996, 5, 1305-1310. A human homologue of Drosophila minibrain (MNB) is expressed in neuronal regions affected in Down syndrome and maps to the critical region) and animals over-expressing DYRK1a have learning and memory deficiencies. (Smith D J, Stevens M E, Sudanagunta S P, Bronson R T, Makhinson M, Watabe A M, O'Dell T J, Fung J, Weier H U, Cheng J F, Rubin E M. Nat Genet. 1997, 16(1):8-9. Functional screening of 2 Mb of human chromosome 21q22.2 in transgenic mice implicates minibrain in learning defects associated with Down syndrome) and may therefore be seen as a possible target for treating learning deficiencies associated with Down's.

In a first aspect there is provided use of a compound or salt or hydroxide thereof of the following formula (I)

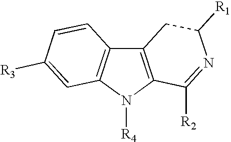

wherein R₁ is selected from hydrogen; substituted or unsubstituted C₁-C₆ alkyl, alkenyl, or alkyloxy, alkylthio, alkyloxycarbonyl; hydroxyl; nitro; amino; halo or oxo;
R₂ and R₃ are independently selected from hydrogen; substituted or unsubstituted C₁-C₆ alkyl, alkenyl, or alkyloxy, alkylthio, alkyloxycarbonyl; hydroxyl; nitro; amino or halo; and
R₄ is selected from hydrogen; substituted or unsubstituted C₁-C₆ alkyl, alkenyl, or alkyloxycarbonyl;
the dashed line represents a single or double bond,
   for the manufacture of a medicament for alleviating or minimising a symptom or condition observed in a Down's syndrome subject.

Typically the compound is used to improve and/or alleviate deficiencies in learning as experienced by subjects with Down's syndrome. Generally, Down's syndrome subjects are born with a brain capacity (IQ) similar to subjects without Down's syndrome, but as they grow, Down's syndrome subjects generally display a slower and/or reduced learning capacity such that by age 13, Down's syndrome subjects display a mean IQ below 50. It is envisaged therefore that the compounds of the present invention may be administered to a Down's syndrome subject from birth, or soon thereafter, until adolescence or possibly into and during adulthood.

In a further aspect there is provided a method of treating or preventing a learning impairment or deficiency in a subject suffering from Down's syndrome, the method comprising administering to the Down's syndrome subject a compound or salt or hydroxide thereof of the following formula (I)

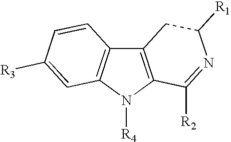

wherein R₁, R₂, R₃ and R₄ and the dashed line have the same meanings as given herein above.

The above method may generally be practised on Down's syndrome subjects from birth to adolescence.

Preferably R₁, R₂ and R₃ are independently selected from hydrogen, hydroxyl or C₁-C₆ alkyl or alkyloxy; and R₄ is selected from hydrogen or C₁-C₆ alkyl.

Typical salts may be formed by preparing a quaternary ammonium salt at one or more of the nitrogen atoms. For example, reaction with an inorganic or organic acid may be used to protonate the nitrogen atom to provide a positively charged nitrogen which is charge balanced with an anion species. Similarly, a hydroxide derivative may be provided by reaction with water.

Particularly preferred compounds are:

Harmine, where R₁ is H, R₂ is CH₃, R₃ is OCH₃, R₄ is H and the dashed line is a double bond;

Harmaline, where R₁ is H, R₂ is CH₃, R₃ is OCH₃, R₄ is H and the dashed line is a single bond;

Harmane, where R₁ is H, R₂ is CH₃, R₃ is H, R₄ is H and the dashed line is a double bond; and

Harmalol, where R₁ is H, R₂ is CH₃, R₃ is OH, R₄ is H and the dashed line is a single bond.

Harmine is an alkaloid from seeds of Peganum harmala L., Zygophyllaceae. It is an active ingredient of hallucinogenic drinks made in the western Amazon region from related plants. There is no known therapeutic use but it was suggested to be a cure for postencephalitic Parkinson disease in the 1920's.

For use according to the present invention, the compounds or physiologically acceptable salt, hydroxide or other physiologically functional derivative thereof described herein may be presented as a pharmaceutical formulation, comprising the compound or physiologically acceptable salt, hydroxide or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersable granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Dosages of the compounds of the present invention include those sufficient to provide the benefical effects for alleviating or minimising a symptom or condition observed in a Down's syndrome subject, whilst not eliciting an hallucinogenic effect on the subject.

### DETAILED DESCRIPTION

### Materials and Methods

### Kinase Assays

All assays (25.5 μl) were carried out at room temperature (21° C.) and were linear with respect to time and enzyme concentrations under the conditions used. Assays were performed for 30 minutes except assays for LCK and PKB which were performed for 15 minutes using Multidrop Micro reagent dispensers (Thermo Electron Corporation, Waltham, Mass. 02454, USA) in a 96-well format. The concentration of magnesium acetate and [γ-³³P] ATP (800 cpm/pmol) in the assays was one of three concentrations (5, 20 or 50 μM) in order to be at or below Km for ATP for the enzyme. Assays were initiated with MgATP and stopped by addition of 5 μl of 0.5M orthophosphoric acid. Assays were then harvested onto P81 filterplates using a unifilter harvester (PerkinElmer, Boston, Mass. 02118, USA), with a wash buffer of 50 mM orthophosphoric acid. Filterplates were then air dried overnight. 10 μl of Microscint O was added per well prior to counting for radioactivity.

MKK1, JNK/SAPK1c, SAPK2a/p38, SAPK2b/p38β2, SAPK3/p38γ, SAPK4/p38δ, MAPK2/ERK2, MAPKAP-K1β, MAPKAP-K2, MSK1, PRAK, PKA, PKCα, PDK1, SGK, p70S6K, GSK3β, ROCK-II, PRK2, AMPK, CHK1, CHK2, CK2, PHK, LCK, were assayed according to Davies et al., (Davies, S. P., Reddy, H., Caivano, M. and Cohen, P. Biochem. J. 351, 95-105, 2000.

CSK and CDK2/cyclinA, were assayed according to Bain et al. (Bain J, McLauchlan H, Elliott M, Cohen P. Biochem J. 371, 199-204, 2003., except that ATP concentrations were either 5, 20 or 50 micromolar so that they were at or below the Km for ATP. The ATP concentrations used were as follows, 5 μM for assays of MKK1, SAPK3, SAPK4, GSK3β, PRK2 CK2 20 μm for assays of JNK, SAPK2β, MAPKAP-K2, MSK1, PRAK, PKA, PKCβ, PDK1, SGK, p70S6K, ROCK-II, CHK1, CHK2, CSK, CDK2, and 50 μM for assays of SAPK2a, MAPK2/ERK2, AMPK, LCK, PHK and MAPKAP-K1β.

The following kinases were assayed against the substrates indicated. Aurora B was assayed against the substrate peptide LRRLSLGLRRLSLGLRRLSLGLRRLSLG (300 μM), ERK8 and MST-2 were both assayed against MBP (0.33 mg/ml). IKKβ was assayed against the substrate peptide LDDRHDSGLDSMKDEEY (300 μM), JNK3 was assayed against 3 μM ATF2[19-96]. MAPKAP-K3 and PKD1 were both assayed against KKLNRTLSVA (30 μM), MARK3 was assayed against CHKtide (KKKVSRSGLYRSPSMPENLNRPR) substrate peptide (300 μM). MNK1 and MNK2 were both assayed against eIF4E protein (0.5 mg/ml). NEK7 was assayed against FLAKSFGSPNRAYKK (300 μM), PIM2 was assayed against RSRHSSYPAGT (300 μM), ΔPH PKBβ-S474D was assayed against Crosstide (30 μM), PLK1 was assayed against ISDELMDATFADQEAKKK (300 μM). PRK2 was assayed against Long S6 peptide, KEAKEKRQEQIAKRRRLSSLRASTSKSGGSQK (30 μM), RSK2 was assayed against KKLNRTLSVA (30 μM), SRC was assayed against cdc2 peptide, KVEKIGEGTYGVVYK, (300 μM) and SRPK1 was assayed against peptide RSRSRSRSRSRSRSR (300 μM). All enzymes were diluted in 50 mM Tris/HCl pH 7.5, 0.1 mM EGTA, 1 mg/ml BSA, 0.1% β-mercaptoethanol buffer and assayed in an incubation containing 50 mM Tris/HCl pH7.5, 0.1 mM EGTA, 0.1% β-mercaptoethanol, CAMK-1 (5-20 mU) diluted in 50 mM Tris/HCl pH 7.5, 0.1 mM EGTA, 1 mg/ml BSA, 0.1% β-mercaptoethanol is assayed against substrate peptide YLRRRLSDSNF in an incubation containing 50 mM Tris/HCl pH 7.5, 0.1 mM EGTA, 0.5 mM CaCl₂, 0.3 μM calmodulin, 0.1% β-mereaptoethanol, 300 μM substrate peptide, 10 mM magnesium acetate and 0.05 mM [³³P-γ-ATP] (500-1000 cpm/pmole). EF2K (5-20 mU) diluted in 50 mM Hepes pH 6.6, 0.1% β-mercaptoethanol, 1 mg/ml BSA is assayed against a substrate peptide RKKFGESKTKTKEFL in an incubation containing 50 mM Hepes pH 6.6, 0.2 mM CaCl2, 0.3 μM Calmodulin, 0.05% b-mercaptoethanol, 300 μM substrate peptide, 10 mM magnesium acetate and 0.05 mM [³³P-γ-ATP] (500-1000 cpm/pmole).

smMLCK (5-20 mU) diluted in 50 mM Hepes pH 7.5, 0.1 mM EGTA, 1 mg.ml BSA, 0.1% β-mercaptoethanol is assayed against substrate peptide KKRPQRATSNVFA in an incubation containing 50 mM Hepes pH 7.5, 0.1 mM EGTA, 5 mM CaCl2, 10 μM calmodulin, 300 μM substrate peptide, 10 mM magnesium acetate and 0.05 mM [³³P-γ-ATP] (500-1000 cpm/pmole).

MAPKAP-K1a (5-20 mU diluted in 20 mM MOPS pH 7.5, 1 mM EDTA, 0.01% Brij35, 5% glycerol, 0.1% β-mercaptoethanol, 1 mg/ml BSA) is assayed against KKLNRTLSVA in a final volume of 25.5 μl containing 50 mM Na-β-glycerophosphate pH 7.5, 0.5 mM EDTA, 30 μM substrate peptide, 10 mM magnesium acetate and 0.05 mM [33P- γ̅ATP] (50-1000 cpm/pmole) and incubated for 40 min at room temperature.

NEK6 (5-20 mU diluted in 50 mM Tris (pH 7.5), 0.1 mM EGTA, 1 mg/ml BSA, 0.1%, β-Mercaptoethanol) is assayed against NEK6 peptide (FLAKSFGSPNRAYKK) in a final volume of 25.5 μl containing 50 mM Tris (pH 7.5), 0.1 mM EGTA, 0.01% Brij, 0.1%, β-Mercaptoethanol, NEK6 peptide (0.3 mM), 10 mM magnesium acetate and 0.05 mM [³³P- γ̅ATP](500-1000 cpm/pmole) and incubated for 30 min at room temperature.

5-20 mU of NEK2a (diluted in 50 mM Tris (pH 7.5), 0.1 mM EGTA, 1 mg/ml BSA, 0.1%, β-Mercaptoethanol) is assayed against NEK2a peptide (RFRRSRRMI) in a final volume of 25.5 μl containing 50 mM Tris (pH 7.5), 0.1 mM EGTA, 0.01% Brij, 0.1%, β-Mercaptoethanol, 300 μM NEK2a peptide, 10 mM magnesium acetate and 0.05 mM [³³P- γ̅ATP](500-1000 cpm/pmole) and incubated for 30 mins at room temperature.

ΔPH-PKBbeta-S474D (5-20 mU diluted in 50 mM Tris pH 7.5, 0.1 mM EGTA, 0.1% β-mercaptoethanol, 1 mg/ml BSA) is assayed against a modified Crosstide peptide (GRPRTSSFAEGKK) in a final volume of 25.5 μl containing 50 mM Tris pH 7.5, 0.05% β-mercaptoethanol, 30 μM substrate peptide, 10 mM magnesium acetate and 0.05 mM [³³P- γ̅ATP] (50-1000 cpm/pmole) and incubated for 30 min at room temperature.

### Abbreviations

Abbreviations used: AMPK, AMP-activated protein kinase; CAM-K, calmodulin-dependent protein kinase; CHK, checkpoint kinase; CK2, casein kinase 2; CREB, cAMP-response element binding protein; CSK, C-terminal Src kinase; DYRK, dual-specificity, tyrosine-phosphorylated and regulated kinase; EF2K elongation factor 2 kinase; EGF, epidermal growth factor; eIF4E eukaryotic initiation factor 4E; ERK, extracellular-signal-regulated kinase; GSK3, glycogen synthase kinase 3; IKKβ. I kappa B kinase ; JNK, c-Jun N-terminal kinase; LCK, lymphocyte kinase; MAPK, mitogen-activated protein kinase; MAPKAP-K1, MAPK-activated protein kinase-1; MAPKAP-K2, MAPK-activated protein kinase 2; MKK, MAPK kinase (also called MEK); MLCK, myosin light chain kinase; MNK, MAPK-integrating kinase; MSK1, mitogen- and stress-activated protein kinase 1; NEK, never in mitosis gene a related protein kinase; PDK1, 3-phosphoinositide-dependent protein kinase 1; PKA, cAMP-dependent protein kinase; PKB, protein kinase B (also called Akt); PKC, protein kinase C; PKG, cGMP-dependent protein kinase; PHK, phosphorylase kinase; PLK, polo-like kinase 1; PRAK, p38-regulated/activated kinase; PRK, protein kinase C-related protein kinase; ROCKII, Rho-dependent protein kinase II; RSK2, ribosomal S6 kinase 2;SAPK2a, stress-activated protein kinase 2a (also called p38); SAPK2b, stress-activated protein kinase 2b (also called p382); SAPK3, stress-activated protein kinase 3 (also called p38γ); SAPK4, stress-activated protein kinase 4 (also called p38δ); SGK, serum- and glucocoiticoid-induced kinase; SRC, v-src sarcoma viral oncogene homologue kinase; SRPK1, serine arginine protein kinase 1; p70S6K, p70 ribosomal protein S6 kinase; Sk, skeletal muscle; Sm, smooth muscle.

### EXAMPLE 1

### Expression and Purification of GST-DYRK1a

Flasks containing 500 ml of LB ampicillin are inoculated with pGEX-DYRK1a and grown at 37° C. until OD at 600 nm is between 0.4-0.6. The culture was then induced with 100 μM IPTG and grown for 14 hours at 26° C.

The culture was harvested by centrifugation at 4,200 rpm for 30 mins and the bacterial pellet resuspended in ice cold lysis buffer (15 ml per 1 L of harvested culture) containing Roche protease inhibitor tablets (one tablet per 25 ml of lysis buffer).

The sample was then sonicated on ice for 8×15 seconds bursts, before being centrifuged at 15,000 rpm for 30 mins.

The clarified lysate was then added to GSH-Sepharose (Pharamacia) [which had been equilibrated in equilibration buffer] and end over end mixed for 45 minutes at 4° C.

The GSH-Sepharose was then washed with wash buffer until the OD at 595 nm was zero.

GST-DYRK1a was eluted from the resin using elution buffer and fractions containing protein are pooled and dialysed overnight into dialysis buffer.

Buffers for purification of GST-DYRK1a

| | |
|---|---|
| Lysis Buffer: | 50 mM Tris/HCl pH7.5 |
| | 150 mM NaCl |
| | 1% Triton |
| | 1 mM EDTA |
| | 1 mM EGTA |
| | 0.1% β-Mercaptoethanol |
| | 0.2 mM PMSF |
| | 1 mM benzamidine |
| Equilibration Buffer/ | 50 mM Tris pH7.5 |
| Wash Buffer: | 250 mM NaCl |
| | 0.03% Brij-35 |
| | 0.1 mM EGTA |
| | 0.1% β-Mercaptoethanol |
| | 0.2 mM PMSF |
| | 1 mM benzamidine |
| Elution: | Wash buffer + 20 mM glutathione (re-pH to 7.5) |
| Dialysis: | 50 mM Tris/HCl pH7.5 |
| | 0.1 mM EGTA |
| | 150 mM NaCl |
| | 50% glycerol |
| | 0.03% Brij-35 |
| | 0.07% β-Mercaptoethanol |
| | 1 mM Benzamidine |
| | 0.1 mM PMSF. |

### EXAMPLE 2

### Kinase Inhibition Assays

DYRK1A 33P screening assay:

DYRK1A (5-20 mU of diluted in 50 mM Tris pH 7.5, 0.1 mM EGTA) was assayed against Woodtide (KKISGRLSPIMTEQ) in a final volume of 25.5 μl containing 50 mM Tris pH 7.5, 0.1 mM EGTA, 350 uM substrate peptide, 10 mM magnesium acetate and 0.5 μl inhibitor and incubated at room temperature for 5 mins, followed by the addition of 0.05 mM [33P-gamma-ATP] (50-1000 cpm/pmole) and incubated for 30 mins at room temperature. Assays were stopped by addition of 5 μl of 0.5 M (3%) orthophosphoric acid then harvested onto P81 Unifilter plates with a wash buffer of 50 mM orthophosphoric acid. Filterplates were then air dried overnight. 10 μl of Microscint O was added per well prior to counting for radioactivity.

DYR2 and DYRK3 were assayed in an analogous manner to that described above for DYRK1a.

Harmine, Harmaline, Harmane and Harmalol were tested at 1 μM and 10 uM (in the assay).

Results are shown in Tables 1 and 2 where values represent the percent activity in relation to the activity obtained in the absence of inhibitor, can be seen that all three compounds are very specific and active at 10 μM and that harmine is a very potent and specific inhibitor of DYRK1a and still shows a high degree of activity at a concentration of 1 μM.

IC50 values were also obtained for DYRK1a, 2 and 3 with harmine (ATP at 50 μM) (i.e. the concentration of the inhibitor that is required to provide 50% inhibition of the enzyme. The results are as follows:

| | | |
|---|---|---|
| DYRK1a | IC50 | 0.08 μM |
| DYRK2 | IC50 | 0.9 μM |
| DYRK3 | IC50 | 0.8 μM. |

The IC50 values were calculated by using half log dilutions of inhibitor, starting at 100 μM.

### Table 1

### Legend

Activities are presented as a percentage of those obtained in control assays carried out in the absence of inhibitor

TABLE 1

| | Harmaline | | Harmane | | Harmalol | | Harmine | |
|---|---|---|---|---|---|---|---|---|
| Concentration (μM) | 1 | +/−SE | 1 | +/−SE | 1 | +/−SE | 1 | +/−SE |
| MKK1 | 47 | 5 | 97 | 3 | 92 | 7 | 76 | 3 |
| JNK/SAPK1c | 99 | 6 | 96 | 4 | 87 | 2 | 85 | 11 |
| JNK3 | 87 | 3 | 75 | 2 | 92 | 5 | 89 | 1 |
| SAPK2a/p38 | 89 | 6 | 95 | 5 | 95 | 1 | 95 | 5 |
| SAPK2b/p38β2 | 91 | 6 | 90 | 7 | 88 | 4 | 94 | 5 |
| SAPK3/p38g | 98 | 0 | 108 | 4 | 98 | 9 | 82 | 3 |
| SAPK4/p38d | 76 | 3 | 94 | 6 | 101 | 0 | 96 | 7 |
| MAPK2/ERK2 | 80 | 2 | 86 | 7 | 88 | 3 | 98 | 2 |
| ERK8 | 58 | 2 | 39 | 3 | 41 | 2 | 25 | 1 |
| MAPKAP-K1a | 98 | 0 | 92 | 3 | 101 | 5 | 105 | 6 |
| MAPKAP-K1b | 87 | 2 | 83 | 7 | 87 | 1 | 89 | 0 |
| MAPKAP-K2 | 104 | 3 | 114 | 3 | 79 | 4 | 78 | 6 |
| MAPKAP-K3 | 79 | 3 | 85 | 3 | 95 | 1 | 89 | 4 |
| MSK1 | 98 | 5 | 98 | 1 | 84 | 3 | 90 | 1 |
| MNK1 | 89 | 6 | 87 | 0 | 80 | 1 | 84 | 3 |
| MNK2 | 88 | 0 | 75 | 9 | 97 | 6 | 84 | 0 |
| PRAK | 88 | 1 | 80 | 2 | 84 | 8 | 81 | 2 |
| PKA | 70 | 0 | 80 | 4 | 90 | 2 | 82 | 2 |
| PKCa | 88 | 2 | 84 | 1 | 109 | 0 | 96 | 0 |
| PDKI | 100 | 1 | 86 | 0 | 88 | 1 | 89 | 2 |
| PKBΔph | 80 | 2 | 83 | 1 | 92 | 6 | 107 | 3 |
| PKBb | 97 | 3 | 92 | 6 | 103 | 3 | 94 | 1 |
| SGK | 14 | 1 | 98 | 2 | 62 | 4 | 66 | 1 |
| CAMK-1 | 99 | 2 | 108 | 2 | 93 | 7 | 107 | 1 |
| smMLCK | 92 | 3 | 93 | 8 | 76 | 4 | 88 | 1 |
| EFK2 | 106 | 9 | 96 | 3 | 97 | 9 | 102 | 1 |
| p70 S6K | 71 | 2 | 81 | 4 | 95 | 3 | 90 | 6 |
| GSK3b | 77 | 5 | 99 | 0 | 90 | 4 | 82 | 7 |
| ROCK-II | 99 | 0 | 80 | 1 | 96 | 4 | 84 | 7 |
| PRK2 | 97 | 1 | 79 | 2 | 86 | 6 | 82 | 0 |
| AMPK | 97 | 3 | 75 | 5 | 92 | 1 | 98 | 1 |
| CHK1 | 78 | 9 | 118 | 5 | 93 | 1 | 86 | 4 |
| CHK2 | 89 | 0 | 89 | 4 | 76 | 5 | 78 | 6 |
| CK1 | 84 | 2 | 82 | 1 | 83 | 1 | 61 | 4 |
| CK2 | 94 | 4 | 96 | 3 | 55 | 7 | 82 | 8 |
| PHK | 73 | 9 | 94 | 6 | 101 | 0 | 103 | 5 |
| Lck | 77 | 4 | 92 | 5 | 85 | 2 | 92 | 2 |
| CSK | 64 | 3 | 92 | 1 | 110 | 2 | 92 | 1 |
| Src | 47 | 0 | 106 | 0 | 101 | 0 | 108 | 2 |
| CDK2/cyclin A | 99 | 9 | 110 | 3 | 97 | 5 | 98 | 5 |
| DYRK1a | 78 | 5 | 39 | 5 | 25 | 1 | 4 | 0 |
| NEK2a | 69 | 3 | 103 | 2 | 104 | 6 | 106 | 11 |
| NEK6 | 99 | 5 | 87 | 4 | 94 | 4 | 96 | 0 |
| NEK7 | 95 | 3 | 100 | 4 | 83 | 0 | 90 | 6 |
| IKKb | 78 | 1 | 90 | 7 | 93 | 1 | 97 | 8 |
| PIM2 | 86 | 5 | 87 | 6 | 90 | 7 | 77 | 2 |
| SRPK1 | 104 | 5 | 100 | 5 | 88 | 0 | 86 | 1 |
| Aurora B | 98 | 0 | 98 | 1 | 110 | 3 | 100 | 9 |
| MARK3 | 99 | 1 | 108 | 5 | 93 | 7 | 86 | 1 |
| MST2 | 98 | 4 | 116 | 5 | 85 | 5 | 91 | 5 |
| PKD1 | 99 | 5 | 103 | 6 | 75 | 3 | 86 | 3 |
| PLK1 | 92 | 4 | 109 | 6 | 88 | 8 | 92 | 4 |

TABLE 2

| | Harmaline | | Harmane | | Harmalol | | Harmine | |
|---|---|---|---|---|---|---|---|---|
| Concentration (μM) | 10 | +/−SE | 10 | +/−SE | 10 | +/−SE | 10 | +/−SE |
| MKK1 | 39 | 3 | 97 | 1 | 66 | 6 | 73 | 4 |
| JNK/SAPK1c | 96 | 3 | 72 | 3 | 84 | 6 | 69 | 7 |
| JNK3 | 81 | 1 | 74 | 2 | 94 | 4 | 83 | 1 |
| SAPK2a/p38 | 86 | 1 | 95 | 3 | 74 | 2 | 81 | 5 |
| SAPK2b/p38β2 | 93 | 4 | 107 | 4 | 79 | 1 | 87 | 3 |
| SAPK3/p38g | 94 | 3 | 92 | 2 | 92 | 8 | 72 | 5 |
| SAPK4/p38d | 113 | 7 | 101 | 3 | 93 | 1 | 82 | 2 |
| MAPK2/ERK2 | 89 | 4 | 93 | 1 | 85 | 3 | 89 | 5 |
| ERK8 | 21 | 2 | 28 | 1 | 21 | 1 | 19 | 1 |
| MAPKAP-K1a | 90 | 1 | 95 | 4 | 85 | 5 | 92 | 1 |
| MAPKAP-K1b | 80 | 1 | 87 | 4 | 67 | 1 | 95 | 4 |
| MAPKAP-K2 | 112 | 2 | 111 | 9 | 69 | 1 | 82 | 5 |
| MAPKAP-K3 | 82 | 2 | 84 | 2 | 88 | 5 | 93 | 1 |
| MSK1 | 111 | 1 | 85 | 2 | 59 | 9 | 88 | 3 |
| MNK1 | 82 | 2 | 69 | 7 | 48 | 4 | 52 | 1 |
| MNK2 | 77 | 1 | 66 | 9 | 71 | 8 | 98 | 1 |
| PRAK | 93 | 2 | 84 | 1 | 78 | 1 | 75 | 3 |
| PKA | 86 | 1 | 89 | 1 | 75 | 5 | 92 | 2 |
| PKCa | 81 | 0 | 93 | 6 | 78 | 1 | 101 | 1 |
| PDK1 | 95 | 0 | 103 | 2 | 96 | 1 | 95 | 1 |
| PKBΔph | 98 | 5 | 95 | 5 | 58 | 3 | 107 | 7 |
| PKBb | 96 | 8 | 97 | 3 | 77 | 4 | 101 | 7 |
| SGK | 84 | 6 | 112 | 3 | 45 | 8 | 62 | 3 |
| CAMK-1 | 110 | 1 | 99 | 2 | 60 | 2 | 94 | 8 |
| smMLCK | 104 | 6 | 97 | 6 | 71 | 2 | 76 | 6 |
| EFK2 | 100 | 1 | 90 | 9 | 85 | 1 | 101 | 0 |
| p70 S6K | 83 | 3 | 87 | 2 | 69 | 5 | 93 | 9 |
| GSK3b | 82 | 7 | 91 | 2 | 73 | 5 | 72 | 1 |
| ROCK-II | 81 | 0 | 64 | 6 | 81 | 1 | 61 | 6 |
| PRK2 | 83 | 3 | 48 | 2 | 54 | 7 | 45 | 0 |
| AMPK | 83 | 2 | 86 | 4 | 52 | 3 | 103 | 4 |
| CHK1 | 67 | 6 | 110 | 7 | 81 | 7 | 86 | 5 |
| CHK2 | 89 | 3 | 87 | 5 | 57 | 5 | 79 | 6 |
| CK1 | 65 | 5 | 73 | 5 | 48 | 2 | 21 | 2 |
| CK2 | 88 | 1 | 61 | 3 | 12 | 1 | 41 | 9 |
| PHK | 88 | 8 | 86 | 3 | 63 | 9 | 82 | 2 |
| Lck | 87 | 2 | 98 | 4 | 68 | 4 | 91 | 5 |
| CSK | 90 | 1 | 107 | 6 | 92 | 1 | 101 | 9 |
| Src | 65 | 5 | 105 | 3 | 83 | 0 | 98 | 6 |
| CDK2/cyclin A | 98 | 5 | 82 | 4 | 77 | 5 | 62 | 9 |
| DYRK1a | 8 | 2 | 11 | 1 | 3 | 0 | 1 | 1 |
| NEK2a | 109 | 2 | 106 | 0 | 83 | 5 | 111 | 7 |
| NEK6 | 87 | 5 | 94 | 7 | 92 | 1 | 99 | 2 |
| NEK7 | 115 | 6 | 96 | 2 | 56 | 9 | 92 | 2 |
| IKKb | 82 | 5 | 76 | 2 | 69 | 5 | 73 | 1 |
| PIM2 | 101 | 4 | 78 | 3 | 57 | 0 | 41 | 1 |
| SRPK1 | 97 | 3 | 105 | 5 | 72 | 11 | 83 | 6 |
| Aurora B | 112 | 1 | 96 | 3 | 83 | 6 | 102 | 1 |
| MARK3 | 103 | 6 | 97 | 8 | 84 | 5 | 86 | 1 |
| MST2 | 105 | 3 | 108 | 2 | 74 | 1 | 81 | 1 |
| PKD1 | 101 | 5 | 71 | 4 | 21 | 4 | 53 | 6 |
| PLK1 | 100 | 9 | 128 | 4 | 81 | 4 | 87 | 3 |

1. A method of alleviating or minimising a symptom or condition observed in a Down's syndrome subject comprising the step of administering to a Down's syndrome subject a therapeutically effective amount of a compound or salt or hydroxide thereof of the following formula (I) 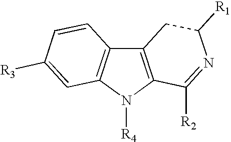
wherein R₁ is selected from hydrogen; substituted or unsubstituted C₁-C₆ alkyl, alkenyl, or alkyloxy, alkylthio, alkyloxycarbonyl; hydroxyl; nitro; amino; halo or oxo;
R₂ and R₃ are independently selected from hydrogen; substituted or unsubstituted C₁-C₆ alkyl, alkenyl, or alkyloxy, alkylthio, alkyloxycarbonyl; hydroxyl; nitro; amino or halo; and
R₄ is selected from hydrogen; substituted or unsubstituted C₁-C₆ alkyl, alkenyl, or alkyloxycarbonyl;
the dashed line represents a single or double bond.

2. The method of claim 1, wherein the compound is administered to a Down's syndrome subject from birth, or soon thereafter, until adolescence or possibly into and during adulthood.

3. The method of claim 1, wherein R₁, R₂ and R₃ are independently selected from hydrogen, hydroxyl or C₁-C₆ alkyl or alkyloxy; and R₄ is selected from hydrogen or C₁-C₆ alkyl.

4. The method of claim 1, wherein the salts are formed by preparing a quaternary ammonium salt at one or more of the nitrogen atoms.

5. The method of claim 1, wherein the compounds are selected from the group consisting of:
(i) Harmine, where R₁ is H, R₂ is CH₃, R₃ is OCH₃, R₄ is H and the dashed line is a double bond;
(ii) Harmaline, where R₁ is H, R₂ is CH₃, R₃ is OCH₃, R₄ is H and the dashed line is a single bond;
(iii) Harmane, where R₁ is H, R₂ is CH₃, R₃ is H, R₄ is H and the dashed line is a double bond; and
(iv) Harmalol, where R₁ is H, R₂ is CH₃, R₃ is OH, R₄ is H and the dashed line is a single bond.

6. A pharmaceutical formulation, comprising a compound or physiologically acceptable salt, hydroxide or other physiologically functional derivative thereof, of the following formula (I) 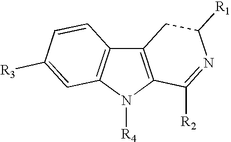
wherein R₁ is selected from hydrogen; substituted or unsubstituted C₁-C₆ alkyl, alkenyl, or alkyloxy, alkylthio, alkyloxycarbonyl; hydroxyl; nitro; amino; halo or oxo;
R₂ and R₃ are independently selected from hydrogen; substituted or unsubstituted C₁-C₆ alkyl, alkenyl, or alkyloxy, alkylthio, alkyloxycarbonyl; hydroxyl; nitro; amino or halo; and
R₄ is selected from hydrogen; substituted or unsubstituted C₁-C₆ alkyl, alkenyl, or alkyloxycarbonyl;
the dashed line represents a single or double bond, together with one or more pharmaceutically acceptable carriers therefore and optionally other therapeutic and/or prophylactic ingredients.

7. The pharmaceutical formulation of claim 6, wherein R₁, R₂ and R₃ are independently selected from hydrogen, hydroxyl or C₁-C₆ alkyl or alkyloxy; and R₄ is selected from hydrogen or C₁-C₆ alkyl.

8. The pharmaceutical formulation of claim 6, wherein the salts are formed by preparing a quaternary ammonium salt at one or more of the nitrogen atoms.

9. The pharmaceutical formulation of claim 6, wherein the compounds are selected from the group consisting of:
(i) Harmine, where R₁ is H, R₂ is CH₃, R₃ is OCH₃, R₄ is H and the dashed line is a double bond;
(ii) Harmaline, where R₁ is H, R₂ is CH₃, R₃ is OCH₃, R₄ is H and the dashed line is a single bond;
(iii) Harmane, where R₁ is H, R₂ is CH₃, R₃ is H, R₄ is H and the dashed line is a double bond; and
(iv) Harmalol, where R₁ is H, R₂ is CH₃, R₃ is OH, R₄ is H and the dashed line is a single bond.

10. The pharmaceutical formulation of any of claim 6, in a form suitable for oral, topical, rectal or parenteral, nasal and/or pulmonary administration.

11. The pharmaceutical formulation of claim 10, wherein when suitable for pulmonary administration via the buccal cavity, particles containing an active compound have a diameter in the range of 0.5 to 7 microns and are delivered to the bronchial tree of the recipient.

12. The method of claim 1, wherein the dosage of the compound is sufficient to alleviate or minimise a symptom or condition observed in a Down's syndrome subject, whilst not eliciting an hallucinogenic effect on the subject.
